(19) United States
(12) Patent Application Publication
(10) Pub. No.: US 2020/0157501 A1
(43) Pub. Date: May 21, 2020

(54) SINGLE BREAST CELL-DERIVED ORGANOIDS

(71) Applicant: Rutgers, The State University of New Jersey, New Brunswick, NJ (US)

(72) Inventor: Hatem Sabaawy, Neshanic Station, NJ (US)

(21) Appl. No.: 16/625,950

(22) PCT Filed: Jun. 28, 2018
(86) PCT No.: PCT/US18/39988

§ 371 Date: Dec. 23, 2019

Related U.S. Application Data
(60) Provisional application No. 62/526,045, filed on Jun. 28, 2017.

(51) Int. Cl. C12N 5/071; G01N 33/50; A61K 49/00

(52) U.S. Cl. CPC C12N 5/0631; G01N 33/5011; A61K 49/0008; C12N 2533/90; C12N 2501/115; C12N 2513/00; C12N 2501/999; C12N 2501/33; C12N 2500/24; C12N 2500/12; C12N 2501/11

(57) ABSTRACT

The present invention relates to organoids derived from a single cell, such as a breast cancer cell, and methods and compositions relating to the production and use thereof, including cell culture medium for producing organoids and methods of personalized treatment for breast cancer. The invention further provides a humanized mouse including a breast organoid derived from a patient's breast cell.

### CROSS-REFERENCE TO RELATED APPLICATION

This application claims the benefit of U.S. Provisional Application No. 62/526,045 filed Jun. 28, 2017, the disclosure of which is incorporated herein by reference in its entirety.

### BACKGROUND OF THE INVENTION

The inability to propagate primary tissues represents a major hurdle to understanding the mechanisms of regeneration and the balance of differentiated cells versus stem cells in adult organisms. A need exists to better understand primary human pathological disorders such as injury repair and tumor development. For cancer studies, current cancer models do not adequately represent the molecular and cellular diversity of human cancers. Existing human cancer cell lines lack defined and detailed information regarding the clinical presentation of the cancer and have inherent limitations for deciphering the mechanisms of therapy resistance. For injury repair, there is a lack of understanding of the mechanisms of regeneration and shortage of tissue and organs for transplantation. Therefore, novel methods to maintain primary tissues for cancer, new drug discovery approaches to treat cancer and regenerative medicine indications are needed.

Maintaining the balance between normal differentiated cells and progenitor or stem cells is complex. Adult stem cells provide regeneration of different tissues, organs, or neoplastic growth through responding to cues regulating the balance between cell proliferation, cell differentiation, and cell survival, with the later including balanced control of cell apoptosis, necrosis, senescence and autophagy. Epigenetic changes, which are independent of the genetic instructions but heritable at each cell division, can be the driving force towards initiation or progression of diseases. Tissue stem cells are heterogeneous in their ability to proliferate, self-renew, and differentiate and they can reversibly switch between different subtypes under stress conditions. Tissue stem cells house multiple subtypes with propensities towards multi-lineage differentiation. Hematopoietic stem cells (HSCs), for example, can reversibly acquire three proliferative states: a dormant state in which the cells are in the quiescent stage of the cell cycle, a homeostatic state in which the cells are occasionally cycling to maintain tissue differentiation, and an activated state in which the cells are cycling continuously. The growth and regeneration of many adult stem cell pools are tightly controlled by these genetic and/or epigenetic responses to regulatory signals from growth factors and cytokines secreted through niche interactions and stromal feedback signals.

Breast cancer (BCa) is the second leading cause of cancer related death in US women. BC is classified clinically into estrogen receptor (ER)+, HER2+, and triple-negative (TNBCs, lacking ER, progesterone receptor (PR) and HER2), with the latter having worse prognosis. BCa patients display a range of genetic, histological and biological heterogeneity, including driver pathway heterogeneity, therefore causing notable disparities in treatment responses. Subsets of TNBCs are characterized by a high frequency of PI3K pathway alterations. The lack of BCa tissues and models that recapitulate BCa heterogeneity and biomarker diversity has hampered progress towards understanding disease progression and lackluster therapeutic responses, even when targeted therapies are available. Cell lines grown in monolayers to test the efficacy of anticancer drugs and genetically engineered mouse models (GEMM) fail to mimic the complexities of BCa microenvironment or reproduce the diverse mechanisms of therapy resistance. Moreover, patient-derived xenografts (PDXs) are expensive, time consuming, tumors undergo selective engraftment, and lack immune cell-tumor regulation. Currently, drug regimens for BCa are chosen based on tumor ER/PR/HER2 assessed in the diagnostic biopsy, and drug effectiveness is determined after weeks of treatment in patients. To provide more effective therapies, primary BCa cells must be propagated from each patient, individual effective doses (monotherapy or combinations) must be determined, and biomarkers and drivers of resistance to therapy must be interrogated in each patient's BCa tissues.

### SUMMARY OF THE INVENTION

In one embodiment, the present invention provides a method of making an organoid from a mammalian breast tissue in vitro comprising: isolating cells from a mammalian breast tissue to provide isolated cells; and amplifying the cells by culturing in an extracellular matrix in an organoid medium for a time sufficient to produce organoids that exhibit endogenous three-dimensional organ architecture.

In another embodiment, the invention provides an in vitro breast organoid comprising epithelial cells and myoepithelial cells, the organoid exhibiting endogenous three-dimensional organ architecture.

In one embodiment, the in vitro breast organoid is derived from a single epithelial cell of a breast tissue, the organoid exhibiting endogenous three-dimensional organ architecture.

In another embodiment, the invention provides an in vitro breast organoid derived from primary breast normal tissue, wherein the organoid comprises epithelial cells and myoepithelial cells and exhibits endogenous three-dimensional organ architecture.

In another embodiment, the invention provides an in vitro breast organoid derived from primary breast cancer tissue, wherein the organoid comprises epithelial cells and myoepithelial cells and exhibits endogenous three-dimensional organ architecture.

In another embodiment, the invention provides an organoid medium supplemented with basic fibroblast growth factor (bFGF), epidermal growth factor (EGF) and hydrocortisone.

In another embodiment, the invention provides a cell culture medium additionally supplemented with Insulin Growth Factor 1 (IGF-1), Insulin, Transferrin and Sodium Selenite.

In another embodiment, the present invention provides a kit including at least one cell culture medium supplemented with bFGF, EGF, hydrocortisone, IGF-1, Insulin, Transferrin and Sodium Selenite.

In another embodiment, the invention provides a method for identifying agents having anticancer activity against breast cancer cells including selecting at least one test agent, contacting a plurality of patient-specific breast organoids derived from the patient's breast cancer cell with the test agent, determining the number of breast organoids in the presence of the test agent and the absence of the test agent, and identifying an agent having anticancer activity if the number or the growth of the organoid cells is less in the presence of the agent than in the absence of the agent. In another embodiment, the method provides a step of treating the patient with the agent identified as having anticancer activity against the patient-specific organoids but not against normal organoids. A method for identifying agents having anticancer activity against breast cancer cells can further include providing a mouse engrafted with breast cancer cells from the patient and containing a tumor formed from the breast cancer cells; administering the identified agent having anticancer activity to the mouse; and determining if the tumor size is reduced in the presence of the identified agent. In another embodiment, a method for identifying agents having anticancer activity against breast cancer cells can further include providing a humanized mouse engrafted with components of a patient's immune system and breast cancer cells from the patient and containing a tumor formed from the breast cancer cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered. This and other embodiments can further include providing a humanized mouse engrafted with breast cancer cells from the patient and containing a tumor formed from the breast cancer cells; administering a control agent to the humanized mouse engrafted with breast cancer cells from the patient; and comparing the size of the tumor in the humanized mouse engrafted with breast cancer cells from the patient to the size of the tumor in the mouse in which the identified agent was administered; and determining if the size of the tumor in the mouse in which the identified agent was administered is reduced relative to the size of the tumor in the humanized mouse engrafted with breast cancer cells from the patient.

In another embodiment, the present invention provides normal patient-specific breast organoids, and methods of using such organoids for personalized therapies for breast cancer, breast tissue replacement after mastectomy and in mammoplasty applications.

In another embodiment, the present invention provides immune humanized mice with implanted patient-specific breast organoids, and methods of using such mice to identify personalized therapies for breast cancer.

In the methods described herein, the organoids exhibit endogenous three-dimensional organ architecture.

### DETAILED DESCRIPTION OF THE INVENTION

In certain embodiments, the present invention provides breast organoids derived in vitro from normal and cancerous tissues, and methods of making and using such organoids, as well as cell culture media and kits. As disclosed in one embodiment herein, certain growth factors in an in vitro environment containing extracellular matrix molecules in a 3-dimensional culture device may be used to make the organoids.

An organoid is a miniature form of a tissue that is generated in vitro and exhibits endogenous three-dimensional organ architecture. See, e.g., Cantrell and Kuo (2015) Genome Medicine 7:32-34. The organoids of the present invention can be used, for example, to: a) determine genomic targets within tumors and prediction of response to therapies in preclinical and clinical trials; b) detect the activity of an anti-cancer agent by examining the number of surviving organoids after treatment; c) detect the activity of a proliferative agent by determining the number of proliferating cells within each organoid and determining gene expression profiling of relevant pathways; d) examine the specificity of agents targeting different cell types within organoids; e) determine the effects of chemotherapy and radiation; f) create mouse models by implantation of the organoid in vivo; g) create preclinical models for examining therapy responses and drug discovery both in vitro and in vivo; and h) determine clonally-targeting anti-cancer therapies.

Accordingly, in one embodiment, the invention provides a method of making an organoid from a mammalian breast tissue in vitro including: isolating cells from a mammalian breast tissue to provide isolated cells; and amplifying one or more of the cells by culturing in an extracellular matrix in an organoid medium for a time sufficient to produce organoids. In one embodiment, the isolated cell are epithelial cells. In one embodiment, a single breast epithelial cell is amplified. One of ordinary skill in the art can determine a time sufficient to induce organoid formation by examining morphological changes associated with organoid formation. In one preferred embodiment, the time sufficient to induce organoid formation is from about five to about fifteen days. In another preferred embodiment, the time sufficient to induce organoid formation is about 14 days.

In one preferred embodiment, the organoid medium includes bFGF, EGF and hydrocortisone. The concentration of bFGF present in the culture medium may range from about 0.1-100 mg/mL (e.g., 1 mg/mL, 5 mg/mL, 10 mg/mL, 15 mg/mL, 20 mg/mL, etc). The concentration of EGF present in the culture medium may range from about 0.1-100 mg/mL (e.g., 1 mg/mL, 5 mg/mL, 10 mg/mL, 15 mg/mL, 20 mg/mL, 25 mg/mL, etc). The concentration of hydrocortisone present in the culture medium may range from about 0.1-10 mM (e.g., 0.1 mM, 0.5 mM, 0.75 mM, 1 mM, 1.5 mM, 2 mM, 5 mM, etc). In a further embodiment, the culture medium further includes one or more of the following: Insulin (1-100 mg/mL), Transferrin (0.5-25 ng/mL), IGF-1 (1.0-50 ng/mL), and Sodium Selenite (0.5-25 ng/mL). In a most preferred embodiment, the culture medium includes the following concentrations: approximately 50 mg/mL Insulin, approximately 5.5 ng/mL Transferrin, approximately 20 ng/mL IGF-1, and approximately 7 ng/mL Sodium Selenite. The culture medium may further include or be substituted with other supplements, growth factors, antibiotics, vitamins metabolites, and hormones, synthetic or natural with similar properties as known in the art. In a preferred embodiment, the culture medium is a commercially available cell culture such as Dulbecco's Modified Eagle Medium (DMEM; Life Technologies), advanced-DMEM (ADMEM) (Life Technologies), or human epithelial growth medium (MEGM™) (Lonza) supplemented with the components described above.

In certain embodiments, the cells are from human breast tissue, and human primary breast cancer tissue. In certain embodiments, cells that may be used to make an organoid are human breast stem-like cells. Such cells are known in the art and may be identified and isolated using markers, for example, CD44^{hi}, CD24^{low}, epithelial-specific antigen (ESA⁺), B38.1⁺ (a Breast/ovarian cancer specific marker), aldehyde dehydrogenase-high (ALDH^{hi}), CD10, EpCam⁺MUC1⁻ and Epcam^{hi}CD49⁺.

In one embodiment, the cells are positive for at least one marker selected from the group consisting of cytokeratin 18 (CK18), basal cytokeratin 14 (CK14), Gross cystic disease fluid protein 15 (GCDFP15), mammoglobin, HER2 (or ERBB2), and MUC1. In another embodiment, the cells are positive for CK18, CK14, GCDFP15 and mammoglobin. In another embodiment, the cells are positive for HER2 and MUC1. Such cells may be identified and isolated by methods of cell sorting that are known in the art. For example, in one embodiment, the cells may be isolated by RNA sorting using methods known in the art, such as molecular beacons and the SmartFlare™ probe protocol (EMD Millipore).

In one preferred embodiment, the cells are obtained from surgically excised tissues by subjecting the tissues to mechanical dissociation, collagenase treatment, and filtration.

In certain embodiments the method is performed with a commercially available extracellular matrix such as Matrigel™. Other extracellular matrices are known in the art for culturing cells. In general, an extracellular matrix comprises laminin, entactin, and collagen. In a preferred embodiment the method is performed using a 3-dimensional culture device (chamber) that mimics an in vivo environment for the culturing of the cells, where preferably the extracellular matrix is formed inside a plate that is capable of inducing the proliferation of stem cells under hypoxic conditions. Such 3-dimensional devices are known in the art. An example of such a device is disclosed by Bansal, N., et al. (2014) Prostate 74, 187-200, the disclosure of which is incorporated herein by reference in its entirety. It has been discovered in accordance with the present invention that the use of a 3-dimensional culture device in a method of making organoids has surprising advantages over other formats, as shown in Table 1.

TABLE 1
Advantages and disadvantages of tested formats

| | Consistency of | | |
|---|---|---|---|
| Format | Organoids | Reproducibility | Efficiency |
| In Matrigel ™ | +++ | +++ | ++++ |
| On Matrigel ™ | + | −−− | ++ |
| Hanging Drop plates | −−− | −−− | −−− |
| Non adherent plate | + | −−− | + |

In another aspect, the invention provides a breast organoid. Two epithelial layers have been morphologically described in the human breast gland: the inner luminal epithelial cell layer and the outer myoepithelial/basal cell layer. The breast organoids of the present invention resemble the structures of the primary tissue. Upon histological and immunofluorescence analyses, one of skill in the art can determine that the organoids recreate the human mammary gland tumor layers of epithelial and fibro-muscular myoepithelial cells. Breast tissue origin of organoids can be confirmed by detecting the expression of mammaglobin and GCDFP15 (cytoplasmic) with outer layer staining with SMA (indicative of myoepithelial cells in organoids).

In another aspect, the invention provides a breast organoid derived in vitro from primary breast cancer tissue. Tumor heterogeneity can be efficiently modeled using the methods described to make an organoid, by mapping the diagnostic dominant clone and tumor subclones from each patient biopsy sample, generating organoids derived from each clone and defining the genetic signature of each clone. A breast organoid derived from primary breast cancer tissue will generally maintain expression of breast lineage-specific markers and the functional secretory profile of the original primary tissue. A breast organoid as described herein can be serially propagated, cryofrozen and regenerated and established as a model for cancer drug discovery and precision therapy.

In another aspect, the invention provides a breast organoid derived in vitro from surgically excised tissues of tumors identified to express histopathological tissue specific and tumorigenic markers. Single cells from these tissues may be isolated with non-contact laser capture microdissection or by RNA sorting, for example using SmartFlare™ probes to generate single cell organoids with known expression features.

The organoids described herein exhibit endogenous three-dimensional organ architecture.

In another embodiment, the invention provides a method for identifying agents having anticancer activity against breast cancer cells from a patient(s) including selecting at least one test agent, contacting a plurality of patient-specific breast organoids derived from the patient's breast cancer cell with the test agent, determining the number of breast organoids in the presence of the test agent and the absence of the test agent, and identifying an agent having anticancer activity if the number or growth of the organoids is less in the presence of the agent than in the absence of the agent. In another embodiment, the method provides a step of treating the patient with the agent identified as having anticancer activity against the patient-specific organoids. A method for identifying agents having anticancer activity can further include providing a mouse engrafted with breast cancer cells from the patient and containing a tumor formed from the breast cancer cells; administering the identified agent having anticancer activity to the mouse; and determining if the tumor size is reduced in the presence of the identified agent.

A method for identifying agents having anticancer activity can further include providing a humanized mouse engrafted with components of a patient's immune system and breast cancer cells from the patient and containing a tumor formed from the breast cancer cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered. In this embodiment, the humanized mice with the patient's immune system can be used to compare the effects of the identified agent (e.g., candidate therapeutic) on tumors in the presence or absence of immune cells to examine a potential role for combination with immunotherapy. These methods can further include providing a humanized mouse (an immune-deficient control mouse) engrafted with breast cancer cells from the patient and containing a tumor formed from the breast cancer cells; administering a control agent to the humanized mouse engrafted with breast cancer cells from the patient; and comparing the size of the tumor in the humanized mouse engrafted with breast cancer cells from the patient to the size of the tumor in the mouse in which the identified agent was administered; and determining if the size of the tumor in the mouse in which the identified agent was administered is reduced relative to the size of the tumor in the humanized mouse engrafted with breast cancer cells from the patient. In this method, if the size of the tumor in the mouse in which the identified agent was administered is reduced relative to the size of the tumor in the humanized mouse engrafted with breast cancer cells from the patient, the identified agent can be confirmed as a successful treatment for cancer in the patient.

In another embodiment, the invention provides a method of selecting a personalized treatment for breast cancer in a subject including: selecting at least one form of treatment, contacting a plurality of breast organoids with the form of treatment, wherein the organoids are derived from breast cancer cells from the subject, determining the number of breast organoids in the presence of the treatment and the absence of the treatment, and selecting the treatment if the number or growth of the breast organoids is less in the presence of the treatment than in the absence of the treatment. Various types of therapy can then be examined using the organoids to determine therapy resistance before initiation, to tailor the therapy for each individual patient based on oncogenic driver expression in the organoids, as well as further study induced clonal selection processes that are the frequent causes of relapse. Various forms, combinations, and types of treatment are known in the art, such as radiation, hormone, chemotherapy, biologic, and bisphosphonate therapy. The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. Terms such as “treating” or “treatment” or “to treat” or “alleviating” or “to alleviate” refer to therapeutic measures that cure, slow down, lessen symptoms of, and/or halt progression of a diagnosed pathologic condition.

The foregoing methods may be facilitated by comparing therapeutic effects in organoids derived from cancer cells and normal cells from the same patient. For example, normal organoids and cancer organoids derived from cells of the same patient can be assessed to determine genetic and epigenetic mutations and gene expression profiles that are cancer-specific, thereby allowing the determination of gene-drug associations and optimization of treatment. Such comparisons also allow one to predict a therapeutic response and to personalize treatment in a specific patient,

In another aspect of this method, clonally targeted therapies can be determined by testing the effect of a therapeutic agent on multiple organoids derived from subsequently determined dominant clones of breast cancer cells identified in the tumor tissue from a patient, and comparing to the effect of the therapeutic agent on organoids derived from normal cells of the same patient.

In another aspect, the invention provides a cell culture medium supplemented with bFGF, EGF, and hydrocortisone. In another embodiment, the invention provides a cell culture medium supplemented with bFGF, EGF, hydrocortisone, IGF-1, Insulin, Transferrin and Sodium Selenite. In a preferred embodiment, the medium is a commercially available breast epithelial cell growth medium such as ADMEM (Life Technologies).

In another aspect, the invention provides kits to make an organoid from a single cell. In an embodiment, a kit contains at least one container for an organoid medium as previously described. The containers may also contain the necessary supplements (growth factors, antibiotics, hormones, vitamins, amino acids, and combinations thereof) for a differentiation medium and an organoid medium. The kit may further include the necessary components for a 3-dimensional culture device, for example, plates, and/or materials for an extracellular matrix, e.g. Matrigel™. The kit may further contain a set of instructions to perform the methods of making an organoid from a single cell as previously described.

In another embodiment, the present invention provides a mouse with an implanted patient-specific breast organoid. In one embodiment, the mouse is a humanized mouse. In another embodiment, the mouse is a human immune system (HIS)-reconstituted mouse. In another embodiment, the mouse is non-obese diabetic (NOD)-Rag (−)-γ chain (−) (NRG) mouse.

Methods of making HIS-reconstituted mice are known in the art and disclosed for example by Drake et al. (2012) Cell Mol Immunol 9:215-24 and Harris et al. (2013) Clinical and Experimental Immunology 174:402-413. In accordance with one aspect of the present invention, human stem cells from patient, for example from a diagnostic bone marrow sample or HLA-matched, are transplanted into neonatal NRG mice to engraft components of the patient's immune system. The mice are later subjected to grafting with breast organoids derived from breast cells of the same patient orthotopically in the mouse fat pad. The mice are useful for identifying new treatments, assessing responses to therapy, and evaluating combination therapies.

The following non-limiting examples serve to further illustrate the invention.

### Example 1

In the experiments described below, 3D patient derived breast organoids (PDBOs) were developed that validate the use of PDBOs from breast tumors which match patient genetic profiles to predict responses to cancer therapy treatments. Patient derived primary cells from resections and biopsies were utilized. Breast tissue-specific culture conditions were established and organoid forming efficiency (OFE) was examined over the extracellular matrix (ECM) Matrigel, and in 3D culture chambers in 3D conditions. Expression of tissue specific markers were correlated in organoids compared to their corresponding cancer type, to utilize tumor type and genetically defined organoids to study cancer progression and therapy responses. The cellular phenotype breast cancer was determined and it was demonstrated that these PDBOs have tissue specific signaling (e.g. ER and cytokeratins expression). These experiments demonstrated that breast cancer organoids could be serially propagated, cryofrozen and regenerated and established as a model for cancer drug discovery and precision therapy. Table 2 below includes the media and culture conditions in a typical embodiment of producing breast organoids.

TABLE 2
Breast Organoid Media

| | | | 3D Culture | |
|---|---|---|---|---|
| Primary | Collection | Process | (Phase II in | Days to |
| Tissue | Media | Time | Matrigel) | organoids |
| Breast | DMEM medium + | Dissociate tissue | ADMEM medium | Direct to Phase |
| | 10% FBS | for 10-12 hours | (preferred) or | II: 14 |
| | Pencillin (3,000 | | MEGM medium + | |
| | Units/mL) + | | bFGF | |
| | Streptomycin | | (10 mg/mL) + | |
| | (300 μg/mL) | | EGF (20 mg/mL) + | |
| | | | Hydrocortisone | |
| | | | (1 mM) + IGF-1 | |
| | | | (20 ng/mL) + | |
| | | | Insulin | |
| | | | (50 mg/mL) + | |
| | | | Transferrin (5.5 | |
| | | | ng/mL) + Sodium | |
| | | | Selenite (7 ng/mL) + | |
| | | | Pencillin (1,000 | |
| | | | Units/mL) + | |
| | | | Streptomycin | |
| | | | (100 μg/mL) | |

### Example 2

A reliable approach was developed to generate three-dimensional (3D) organoids from patient-derived cells (patient-derived organoids (PDOs)) with ˜90% efficiency, and these organoids were utilized to improve targeting of AKT with chemoradiation and predict responses to personalized therapies. Cell culture in 3D offers a vast improvement over monolayer culture, as it recreates cell-cell and cell-ECM interactions that affect phenotypes, gene expression and multiple cellular functions. Here, a comprehensive personalized approach of combining clinical BCa data with assessing agent doses based on matching genomic profiles with target engagement in the 3D organoid biological readout system was developed to maximize the potential of therapy success.

As a first step towards generating PDBOs from BCa tissues, multiple parameters were examined, including time and length of culture, number of cells, media, additives and culture conditions. Different culture media tested included DMEM, ADMEM, and human MEGM. The MCF7 and MDA-MB-231 cell lines established from BCa were used for these initial studies. Multiple cell numbers including single BCa cells were seeded at different clonal densities (100 or 500 cells/well) in each medium without serum in pure multilayer matrigel chambers. In vitro 3D cultures have been used for growth of normal mammary epithelium, but never achieved before from primary BCa tissues.

BCa organoids should recreate the human mammary gland tumor layers of epithelial and fibro-muscular myoepithelial cells. First utilized were MCF7 BCa cells to determine best conditions for generating organoids. The optimum media (ADMEM) was identified that allowed growth of organoids from single cells within 13 days (d). The OFE was found to improve by the addition of IGF-1 and Insulin Transferrin Sodium Selenite (ITS) to the culture media that was previously used for generating prostate cancer organoids. The effects of IGF1 and ITS addition resulted from increasing the expression of stem cell factors. Therefore, the optimum growth conditions to produce the highest OFE were determined. These conditions were then utilized to make organoids from primary BCa. Under an IRB-approved protocol, specimen from high-risk BCa cases were collected and processed within 15 minutes of surgery. Areas containing tumor as deemed by the pathologist and normal epithelial counterpart were microdissected. First developed was a 3D culture system fit for growth of mammary cells by isolating epithelial cells microdissected from primary BCa specimens. Qualified pathologists confirmed their BCa origin from the corresponding H&E and IHC staining. Cells were placed in 3D droplet culture chambers containing Matrigel, to mimic the basal lamina of the normal mammary gland, and epithelial mesenchymal growth factors in conditions that permits cellular self-organization of organoid forming cells. BCa cells were embedded as single cells in 3D-well plates. Organoid formation was then followed microscopically daily for two full weeks. Whether these 3D culture conditions are optimized for maintenance of expression of the breast lineage-specific markers and their functional secretory profile was examined. Next, these media were tested directly on primary BCa cells from tumor and normal adjacent tissue (NAT) from mastectomy tissues, and observed that the OFE was superior when cells were grown in ADMEM rather than MEGM. Pathological examination has always confirmed that the tissues dissociated were from BCa cancer foci (>90% tumor), suggesting that normal cell overgrowth is improbable. Nevertheless, to exclude this possibility, single epithelial cells were isolated from both NAT and cancer tissues and their respective OFE evaluated. The OFE of cancer foci-derived cells was significantly higher compared to that of normal tissue-derived cells. Expression of the BCa biomarker (ERBB2 or HER2) was nearly a thousand-fold higher in tumor vs. normal tissues, confirming tumor cell identity. Also, expression of breast lineage-specific markers such as MUC1 was significantly higher in tumor tissue.

It was found that the addition of Insulin, Transferrin, Sodium Selenite and IGF-1 to the basal conditions previously used for 3D culture enhances BCa growth factor signaling, leading to the formation of single cell derived BCa organoids in 14 days, a new finding that by itself could have major impact since drugs inhibiting IGF-1 pathway are commercially available and may be used for breast cancer treatment. The conditions examined included culturing primary BCa cells directly into 3D chambers (directly into Stage II). This resulted in the formation of organoids with multiple different cell phenotypes. These BCa PDBOs continue to grow when the matrigel chamber is large enough (as demonstrated for PDBOs imaged on day 29). Direct 3D culture in stage II was more permissible to forming organoids from BCa tissue than NAT. Data utilizing q-PCR and IF assays confirming the full tissue specific signaling and characterization of breast cancer organoids was also generated. To further elucidate that BCa organoids are derived from BCa stem like cells and generate the different cell types present in breast tissues, PDBOs were generated and fixed at day 14 for IHC analysis using BCa specific markers. Characterization of normal stem cell subtypes in human breast tissues has been challenging. Three epithelial cell types have been morphologically described in the human breast duct: the inner luminal cells and the outer basal and myoepithelial cells. This limited understanding of the progenitor and differentiated cell types comprising the breast ducts that has precluded the development of a normal cell type-based classification system. While there has been more recent interest in normal breast cell subtypes, this research has been difficult to correlate with existing human breast cancer phenotypes. Numerous markers have been used to describe normal human mammary stem/progenitor cells, including CD44^{hi}CD24^{low}, aldehyde dehydrogenase-high (ALDH^{hi}), CD10⁺, EpCAM⁺MUC1⁻ and EpCAM^{hi}CD49⁺. Whether these stem/progenitor cell markers identify the same cell populations remains unknown. There is increasing evidence for the existence of a differentiation hierarchy in the adult human mammary gland. Mammary stem/progenitor cells could give rise to mature epithelium of either the luminal or myoepithelial lineage via a series of lineage-restricted intermediates. The luminal lineage can be further subdivided into ductal and alveolar luminal cells that line the ducts and constitute the alveolar units that arise during pregnancy, respectively.

In contrast, myoepithelial cells are specialized, contractile cells located at the basal surface of the epithelium adjacent to the basement membrane. Cytokeratins such as CK18 are expressed in luminal, but not in myoepithelial cells. In contrast, smooth muscle actin (SMA) and p63 are expressed in all myoepithelial cells, but not in luminal cells.

In the first set of IHC studies, a dual IHC assay was utilized to confirm the expression of BCa specific markers, together with myoepithelial marker SMA and ER, first in the original primary BCa formalin fixed paraffin embedded (FFPE) tissue, then in PDBOs. The breast tissue origin of primary tissue and organoids was confirmed by detecting the expression of mammaglobin and GCDFP15 with a specific antibody cocktail (Cell Marque, CMC90640040). GCDFP15 (also called BRST2) is a 15 kDa glycoprotein which is localized to the apocrine metaplastic epithelium lining breast cysts. GCDFP15 positive staining has a positive predictive value and specificity for the detection of breast cancer of 99%. Mammaglobin is a 10 kDa breast cancer-specific glycoprotein whose overexpression was identified in breast adenocarcinoma compared with normal breast tissue. To demonstrate that the clonally proficient single cells generate differentiated breast cells, the organoids were examined for markers of luminal, basal and differentiated cells, including assessing the breast epithelium lineage-specific markers CK18, CK14 and breast specific GCDFP15 and mammaglobin. The breast tissue origin of organoids was confirmed by detecting the expression of mammaglobin and GCDFP15 with outer layer staining with SMA (indicative of myoepithelial cells in organoids). Other organoids showed expression of ER together with luminal CK8/18. Additionally, a dual IHC assay was utilized to confirm the expression of breast specific ER, together with luminal CK8/18 in the original primary breast cancer FFPE tissue and BCa organoid. The same PDBO was also positive for breast cancer specific GCDFP15 and mammaglobin, further confirming the breast cancer origin of organoids. These data suggest that a functional breast organotypic cultures comprised of multiple cell types including epithelial and myoepithelial cells to more appropriately model signaling and cell-cell interactions in an environment like complex human in vivo breast tissue has been developed.

### Example 3

A PI3K activity score was developed from primary BCa tissues for organoid drug sensitivity studies. Assessing the extent of PI3K pathway activity in BCa is vital for predicting sensitivity to PI3K-targeting drugs, but the best biomarker of PI3K pathway activity in FFPE tumor specimens and organoids is unclear. Here, an IHC-based assay was developed to measure PI3K and DNA damage repair (DDR) pathway activation, that could be used with organoids for testing of targeted therapy for selection and in clinical trials. Tissue from 35 women with BCa, was examined using multiple pathway nodes that include PTEN, INPP4B, pAKT, pS6, and stathmin for PI3K activity and 53BP1 and γH2AX for DDR activity. Based on these markers, an 11-point score of PI3K activation or a 5-point score of DDR activation were created using the combined intensity of the 5- or 2-markers and analyzed in association with proliferation (Ki67), apoptosis (TUNEL), and ER/PR/HER2 status, as well as pathologic features and cancer-specific outcomes. All interpretation of IHC was performed blinded to outcomes. Slides were stained using an automated Ventana system to ensure reliable clinical grade staining. TMA slides were digitally scanned and analyzed with a semiautomated image analysis software system. After appropriate thresholding for each TMA, image analysis was performed to generate the following variables: percentage of nuclei positive (Ki67) and average percentage of cytoplasm staining per cell. For pAKT, pS6, and stathmin, a continuous value was obtained after averaging across the replicate core. Representative images for stained sections were demonstrated from two primary BCa compared to normal mammary gland tissues. Thus, a reliable assay was developed to determine PI3K and DDR activation scores and responses to targeted therapy for selecting effective doses for PI3K and radiation therapy in BCa organoids. These data allow for the conclusion that BCa organoids and a biological system for examining targeted therapy in BCa patient tissues have been generated.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein by reference in their entireties.

We claim:

1. A method of making an organoid from a mammalian breast tissue in vitro comprising: isolating cells from a mammalian breast tissue to provide isolated cells; and amplifying one or more of the cells by culturing in an extracellular matrix in an organoid medium for a time sufficient to produce organoids that exhibit endogenous three-dimensional organ architecture.

2. The method of claim 1 wherein the organoid medium comprises basic fibroblast growth factor (bFGF), epidermal growth factor (EGF) and hydrocortisone.

3. The method of claim 2 wherein the organoid medium further comprises one or more of Insulin, Transferrin, IGF-1, and Sodium Selenite.

4. The method of claim 1 wherein the mammalian tissue is a human tissue.

5. The method of claim 4 wherein the human tissue is human breast tissue.

6. The method of claim 5 wherein the human breast tissue is primary human normal breast tissue, or primary human breast cancer tissue.

7. The method of claim 1 wherein the organoids comprise epithelial cells and myoepithelial cells.

8. The method of claim 1 wherein the time sufficient to produce organoids is about fourteen days.

9. The method of claim 1 wherein the organoid medium is changed every other day.

10. The method of claim 2 wherein the bFGF is present at a concentration of about 1-50 mg/mL.

11. The method of claim 2 wherein the bFGF is present at a concentration of about 1-50 mg/mL, the EGF is present at a concentration of about 1-50 mg/ML, and the hydrocortisone is present at a concentration of about 0.1-10 mM.

12. The method of claim 3 wherein the medium comprises Insulin at a concentration of about 1-100 mg/mL, Transferrin at a concentration of about 0.5-25 ng/mL, IGF-1 at a concentration of about 1.0-50 ng/mL, and Sodium Selenite at a concentration of about 0.5-25 ng/mL.

13. The method of claim 1 wherein the isolated cells are sorted for the presence of at least one marker selected from the group consisting of CK18, CK14, GCDFP15⁺ and mammoglobin.

14. A breast organoid comprising epithelial cells and myoepithelial cells, the organoid exhibiting endogenous three-dimensional organ architecture.

15. A breast organoid derived in vitro from primary breast normal tissue, wherein the organoid comprises epithelial cells and myoepithelial cells and exhibits endogenous three-dimensional organ architecture.

16. A breast organoid derived in vitro from primary breast cancer tissue, wherein the organoid comprises epithelial cells and myoepithelial cells and exhibits endogenous three-dimensional organ architecture.

17. A cell culture medium supplemented with bFGF, EGF and hydrocortisone.

18. A cell culture medium supplemented with Insulin, IGF-1, Transferrin and Sodium Selenite.

19. The cell culture medium of claim 17 further comprising Insulin, IGF-1, Transferrin and Sodium Selenite.

20. A kit comprising the cell culture medium of claim 19.

21. A method for identifying an agent having anticancer activity against breast cancer cells from a patient comprising selecting at least one test agent, contacting a plurality of breast organoids derived from breast cancer cells from the patient with the test agent, determining the number of breast organoids in the presence of the test agent and the absence of the test agent, and identifying an agent having anticancer activity if the number or growth of the organoids derived from breast cancer cells from the patient is less in the presence of the agent than in the absence of the agent.

22. A method of personalized treatment for breast cancer in a subject comprising: selecting at least one form of treatment, contacting a plurality of breast organoids with the form of treatment, wherein the organoids are derived from breast cancer cells from the subject, determining the number of breast organoids in the presence of the treatment and the absence of the treatment, and selecting the treatment if the number or growth of the breast organoids is less in the presence of the treatment than in the absence of the treatment.

23. The method of claim 22 further comprising treating the subject with the selected treatment.

24. A method of personalized treatment for breast disorders in a subject comprising: selecting normal breast cells to generate organoids, wherein the organoids are derived from breast normal cells from the subject, or HLA-matched donors, generating normal patient-specific or HLA-matched breast organoids, and using such organoids for personalized therapies for breast tissue replacement after mastectomy and in mammoplasty applications.

25. A humanized mouse engrafted with components of a patient's immune system and comprising a breast organoid derived from the patient's breast cell grafted into the mouse.

26. The method of claim 21, further comprising providing a mouse engrafted with breast cancer cells from the patient and containing a tumor formed from the breast cancer cells; administering the identified agent having anticancer activity to the mouse; and determining if the tumor size is reduced in the presence of the identified agent.

27. The method of claim 21, further comprising providing a humanized mouse engrafted with components of a patient's immune system and breast cancer cells from the patient and containing a tumor formed from the breast cancer cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered.

28. The method of claim 21 or 27, further comprising providing a humanized mouse engrafted with breast cancer cells from the patient and containing a tumor formed from the breast cancer cells; administering a control agent to the humanized mouse engrafted with breast cancer cells from the patient; and comparing the size of the tumor in the humanized mouse engrafted with breast cancer cells from the patient to the size of the tumor in the mouse in which the identified agent was administered; and determining if the size of the tumor in the mouse in which the identified agent was administered is reduced relative to the size of the tumor in the humanized mouse engrafted with breast cancer cells from the patient.

29. The method of any one of claims 21-24 and 26-28, wherein the organoids exhibit endogenous three-dimensional organ architecture.
